(19) United States
(12) Patent Application Publication
(10) Pub. No.: US 2014/0274741 A1
(43) Pub. Date: Sep. 18, 2014

(54) METHODS TO CAPTURE AND SEQUENCE LARGE FRAGMENTS OF DNA AND DIAGNOSTIC METHODS FOR NEUROMUSCULAR DISEASE

(71) Applicant: The Translational Genomics Research Institute, Phoenix, AZ (US)

(72) Inventors: Jesse M. Hunter, Phoenix, AZ (US); Lisa Baumbach-Reardon, Phoenix, AZ (US)

(73) Assignee: The Translational Genomics Research Institute, Phoenix, AZ (US)

(21) Appl. No.: 14/217,266

(22) Filed: Mar. 17, 2014

Related U.S. Application Data
(60) Provisional application No. 61/791,405, filed on Mar. 15, 2013.

(51) Int. Cl. C12Q 1/68

(52) U.S. Cl. CPC C12Q 1/6874; C12Q 1/6883
USPC 506/2

(57) ABSTRACT

The present invention provides methods of sequencing a large fragment of DNA by hybridizing a set of specifically designed probes to the DNA, shearing the DNA, and sequencing the DNA with Next Generation Sequencing. The probes are designed to target genes of interest at intervals to allow the capture of relatively large DNA fragments. The present invention also provides methods of diagnosing a neuromuscular disease (NMD) comprising detecting mutations in one or more of SCML2, CHRND, OFD1, DYNC1H1, COL6A3, EMD, ARHGAP4, FLNA, MID1IP1, MID1, and CFP.

### CROSS-REFERENCE TO RELATED APPLICATIONS

The present application claims priority to U.S. Application No. 61/791,405, filed Mar. 15, 2013, the entire contents and disclosure of which are herein incorporated by reference thereto.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 1,231,079 byte ASCII (text) file named “Seq_List” created on Mar. 17, 2014.

### TECHNICAL FIELD

The present invention relates to comprehensive genetic testing and discovery of novel mutations for diseases including neuromuscular diseases. The invention also provides methods of diagnosing neuromuscular disease by detecting genetic mutations in biological samples.

### BACKGROUND

There are many types of rare genetic disorders. In order to make an accurate genetic diagnosis of the cause of disease, genetic testing must be done. There are many types of genetic testing. While sequencing genes, known disease causing mutations can be identified. More importantly, new disease causing mutations can be discovered. Discovery of new mutations that lead to disease can uncover critical knowledge important for testing for the disease and discovering, developing, and administering treatment for the disease and even for unrelated disease. There is a need for more effective methods of capturing DNA from patient samples and sequencing selected regions to identify and discover mutations leading to disease.

Spinal muscular atrophies (SMA) are a group of rare inherited disorders characterized by degeneration of lower motor neurons. Motor neurons are the biological wires that connect the spinal cord to muscles. Motor neurons control our voluntary and involuntary muscles, and loss of motor neurons impairs the communication between the brain and muscles resulting in muscle weakness (hypotonia) and atrophy, the primary clinical feature of SMA. With SMA, muscle weakness (hypotonia) usually appears in early childhood and is often apparent at birth. When a child has symptoms of SMA, genetic testing for mutations in the SMN1 gene (the only clinically available test available in the U.S.) accounts for 70-80% of SMA. However, for the other 20-30% of SMA patients who do not have mutations in SMN1, the genetic cause of their disease often remains undiagnosed due to the lack of a clinical test. The heterogeneous etiology of genetic alterations that can result in clinical manifestations of hypotonia has only recently been appreciated. There is a pressing need for accurate and effective methods to identify the genetic mutations causing this and other diseases. Such methods would increase the chances for early diagnosis and better therapeutic treatment of such disease.

### SUMMARY

The present invention provides a method of sequencing a large fragment of DNA, the method comprising a) isolating genomic DNA from a biological sample; b) hybridizing the genomic DNA with a set of probes to form genomic DNA-probe complexes, wherein the set of probes targets sequences across the large fragment of DNA at intervals; c) purifying the genomic DNA from the complexes with affinity chromatography; d) shearing the genomic DNA to produce small fragments of DNA, wherein the small fragments of DNA comprise coding and non-coding sequences from the large fragment of DNA; and e) sequencing the small fragments of DNA with Next Generation Sequencing (NGS) to obtain the sequence of the large fragment of DNA.

The present invention also provides a method of sequencing a large fragment of DNA, the method comprising a) isolating genomic DNA from a biological sample; b) hybridizing the genomic DNA with a first set of probes to form genomic DNA-probe complexes with a portion of the genomic DNA encoding a pseudogene, wherein the set of probes targets sequences across the pseudogene at intervals; c) removing the portion of the genomic DNA encoding the pseudogene with affinity chromatography; d) hybridizing the genomic DNA with a second set of probes to form genomic DNA-probe complexes, wherein the second set of probes targets sequences across the large fragment of DNA at intervals; e) purifying the genomic DNA from the complexes with affinity chromatography; f) shearing the genomic DNA to produce small fragments of DNA, wherein the small fragments of DNA comprise coding and non-coding sequences from the large fragment of DNA; and g) sequencing the small fragments of DNA with Next Generation Sequencing (NGS) to obtain the sequence of the large fragment of DNA.

In some embodiments, the large fragment of DNA includes at least one gene. The pseudogene and the at least one gene may share at least 60%, 70%, 80%, or 90% sequence homology. In certain aspects, the series of probes target intervals across the pseudogene and/or the large fragment of DNA of about 500 bp to about 5,000 bp. In other aspects, the large fragment of DNA comprises at least 1 gene, at least 5 genes, at least 25 genes, at least 50 genes, at least 100 genes, at least 150 genes, at least 200 genes, or at least 250 genes.

In another embodiment, the present invention provides a method of diagnosing a neuromuscular disease (NMD) in a subject, the method comprising a) obtaining a biological sample from the subject; b) isolating genomic DNA from the biological sample; c) sequencing in the genomic DNA at least one gene selected from the group consisting of SCML2, CHRND, OFD1, DYNC1H1, COL6A3, EMD, ARHGAP4, FLNA, MID1IP1, MID1, and CFP; and d) diagnosing NMD in the subject if there is a mutation in the at least one gene. The mutation may be any one of ASN76SER in SCML2, MET1VAL start loss in SCML2, ASP161ASN in CHRND, 1 DNA base pair frameshift deletion at genomic position chromosome 2 position 233398958 in CHRND, GLU958LYS in OFD1, TRP1208LEU in DYNC1H1, LYS2483GLU in COL6A3, a DNA substitution of G to T at the splice junction of EMD at genomic position chromosome X position 153608155, PRO635LEU in ARHGAP4, VAL584LEU in FLNA, ARG655HIS in FLNA, ASP51ASN in MID1IP1, PRO667LEU in MID1, and CYS337TYR in CFP. In certain aspects, the method further comprises administering an effective amount of a therapeutic agent to the subject diagnosed with an NMD.

### BRIEF DESCRIPTION OF THE DRAWINGS

Illustrative and exemplary embodiments of the invention are shown in the drawings in which:

FIG. 1 shows the steps involved in standard fragment capture versus large fragment capture and underscores the advantages of lower cost and greater sequencing coverage available with large fragment capture.

FIG. 2 shows high confidence variants identified in patients suffering from various forms of neuromuscular disease.

FIG. 3 shows low confidence best candidate variants identified in patients suffering from various forms of neuromuscular disease.

### DETAILED DESCRIPTION

As used herein, the term “neuromuscular disease” or “NMD” means any disorder that results in a loss of muscle function. Loss of muscle function can be a result of disruption of the cellular and biological function or structure of muscle tissue or the loss of nervous system function that controls muscle function. Nervous system defects include loss of central nervous system function controlling muscle movements, spinal neuron dysfunction, spinal motor neuron dysfunction, or neuromuscular junctions. Nervous system defects can also result from loss of glial cell function that supports neuronal function.

As used herein, the term “variant” means a DNA base that differs between two people

The term “mutation” means a variant (generally very rare in the population) that is the genetic cause of a disease.

As used herein, a “pseudogene” is section of genomic DNA that is an imperfect copy of a functional gene and has lost its protein-coding ability or is otherwise no longer expressed in the cell. A pseudogene may have a high degree of homology or identity to its functional counterpart. In some aspects, the method of the present invention comprises hybridizing the genomic DNA with a first set of probes to form genomic DNA-probe complexes with a portion of the genomic DNA encoding a pseudogene, wherein the set of probes targets sequences across the pseudogene at intervals. In some embodiments, the pseudogene shares at least 60%, at least 65%, at least 70%, at least 75%, at least 80%, at least 85%, at least 90%, or at least 95% homology with a functional gene. In other embodiments, the pseudogene shares at least 40%, at least 45%, at least 50%, at least 55%, at least 60%, at least 65%, at least 70%, at least 75%, at least 80%, at least 85%, at least 90%, or at least 95% identity with a functional gene.

The present invention relates to a neuromuscular disease (NMD) gene capture panel and large fragment capture techniques. The disclosed large fragment capture techniques confer unique advantages that are not available with standard exome capture and sequencing techniques.

In one embodiment, panels of genes (e.g., NMD genes) are used to develop unique capture probes for these genes. These capture probes are designed in a unique way in that probes are spaced at equal intervals across the entirety of each gene. In one aspect, the spacing between probes is about 500 bp to about 5000 bp, e.g., about 500 bp and another about 2000 bp. In certain specific embodiments, interval is optimized to create the greatest capture efficiency. These unique probes are used in a novel capture process where large fragments of DNA are captured.

There are several benefits to capturing large DNA fragments:
1) Fewer probes can be used to capture sequences of interest decreasing the cost of generating capture probes compared to current techniques that have little or no spacing between probes and even overlap to a large extent.
2) Capture of large continuous fragments is more amenable to identifying allelic variants (phasing) and may be more amenable to newer sequencing technologies being developed where large continuous fragments are needed for sequencing.
3) Capture efficiency is increased by having multiple probes attached to a single piece of DNA.
4) All regions of the entire gene will be sequenced rather than just the coding regions of the gene.

To further explain these advantages, current capture techniques are designed to have one probe hybridize to one short segment of DNA that is subsequently captured by an affinity interaction such as a streptavidin interaction. The capture technique of the present invention is designed to have multiple probes for one large segment of DNA. While not wishing to be bound by any theory, it is thought that having multiple probes hybridized to one large DNA fragment should increase affinity interaction because the affinity interactions are “quasi intramolecular” interactions rather than independent affinity interactions.

While most disease causing mutations are found in coding regions, some mutations are found in intronic regions. These intronic mutations cannot be identified by exome sequencing and can only be identified by whole genome sequencing. Intronic mutations such as inversions, translocations, and deletions can be just as damaging as coding mutations.

In some embodiments, probes to mitochondrial genes are excluded from the probe design because mitochondrial DNA is so prevalent that it unavoidably contaminates all sequencing techniques currently available resulting in sequencing of the entire mitochondrial genome regardless of capture and sequencing techniques used. In other embodiments, probes to these mitochondrial genes are included in the panel.

To further explain the advantages of the present invention, a custom gene capture and sequencing panel (e.g., for sequencing NMD genes) costs about the same amount as sequencing a whole exome. It provides a benefit to patients that cannot be obtained by whole exome sequencing as described above. The panel can be also be used in conjunction with whole exome sequencing or whole genome sequencing if desired, but with increased coverage of NMD genes.

In certain embodiments, the present invention allows for the exclusion of pseudogenes in targeted sequencing. Just as selective capture of large fragments can be used to distinguish between closely related gene sequences, this same technique can be adapted to preferentially remove pseudogene sequences. Pseudogenes are inactive genes that share very high homology with functional genes. This homology results in decreased alignment efficiencies and false positive variant calls. There are thousands of pseudogenes decreasing the ability to identify disease causing variants in many hundreds of genes from targeted panel sequencing, whole exome sequencing, and whole genome sequencing results. This approach is applicable for sequencing genes involved in any disease.

The present invention allows for the capture of a comprehensive list of genes. Overlap between clinical phenotype generally does not allow for sufficient information for a physician to successfully choose which gene or small panel of genes is the correct one to sequence to identify the disease causing mutation. Whole exome sequencing allows for the analysis of many genes, but only for exons. Whole genome sequencing covers introns and exons, but at much higher cost. The largest panels that have been previously analyzed are about 20-200 genes. By making it much more likely for a disease causing gene to be discovered by using such a large comprehensive panel, the present invention provides a significant advantage over other techniques. The present invention also allows for the capturing of a relatively large set of complete genes with relatively few probes.

In some embodiments, the large fragment of DNA comprises at least 1 gene, at least 5 genes, at least 25 genes, at least 50 genes, at least 100 genes, at least 150 genes, at least 200 genes, or at least 250 genes.

The term “sample” as used herein means a sample of biological tissue or fluid or an excretion sample that comprises nucleic acids. Such samples include, but are not limited to, tissue or fluid isolated from subjects. Biological samples may also include sections of tissues such as biopsy and autopsy samples, frozen sections, formalin fixed and paraffin embedded tissue samples, blood, plasma, serum, sputum, stool and mucus. Biological sample also refers to metastatic tissue obtained from, but not limited to, organs such as liver, lung, and peritoneum. Biological samples also include explants and primary and/or transformed cell cultures derived from animal or patient tissues. Biological samples may also be blood, a blood fraction, gastrointestinal secretions, or tissue sample. A biological sample may be provided by removing a sample of cells from an animal, but can also be accomplished by using previously isolated cells (e.g., isolated by another person, at another time, and/or for another purpose), or by performing the methods described herein in vivo. Archival tissues, such as those having treatment or outcome history, may also be used.

As used herein, a “sample” or “biological sample” refers to a sample of biological tissue, fluid or excretion that comprises nucleic acids (e.g., mRNA). It should be noted that a “biological sample obtained from the subject” may also optionally comprise a sample that has not been physically removed from the subject. In some embodiments the sample obtained from the subject is a body fluid or excretion sample including but not limited to seminal plasma, blood, serum, urine, prostatic fluid, seminal fluid, semen, the external secretions of the skin, respiratory, intestinal, and genitourinary tracts, tears, cerebrospinal fluid, sputum, saliva, milk, peritoneal fluid, pleural fluid, peritoneal fluid, cyst fluid, lavage of body cavities, broncho alveolar lavage, lavage of the reproductive system and/or lavage of any other organ of the body or system in the body, and stool.

Numerous well known tissue or fluid collection methods can be utilized to collect the biological sample from the subject in order to determine the expression level of the biomarkers of the invention in said sample of said subject.

Examples include, but are not limited to, blood sampling, urine sampling, stool sampling, sputum sampling, aspiration of pleural or peritoneal fluids, fine needle biopsy, needle biopsy, core needle biopsy and surgical biopsy, and lavage. Regardless of the procedure employed, once a biopsy/sample is obtained the level of the biomarkers can be determined and a diagnosis can thus be made. Tissue samples are optionally homogenized by standard techniques e.g. sonication, mechanical disruption or chemical lysis. Tissue section preparation for surgical pathology can be frozen and prepared using standard techniques. In situ hybridization assays on tissue sections are performed in fixed cells and/or tissues.

In a one embodiment, blood is used as the biological sample. If that is the case, the cells comprised therein can be isolated from the blood sample by centrifugation, for example.

In certain aspects, the methods of the present invention use commercially available reagents and resources. However, the unique ordering of the steps and the reduced number of probes required to capture DNA regions of interest confer superior advantages over know methods of DNA capture and sequencing.

In certain aspects, the present invention encompasses the design of probes specific to genes of interest. The beginning and end coordinates of each gene of interest (for example, in the case of NMD genes totaling 1387 gene regions) can be obtained from available online databases. Genes that overlap or that are within 1000 bp of each other are merged into single regions. The total size (in base pairs) of all regions is summed then divided by the number of probes desired resulting in the interprobe distance. Each region is then divided by the interprobe distance to determine the number of probes for that region. Coordinates for probes are calculated based on beginning and end coordinates for each region and the interprobe distance. In some embodiments, probes are designed and manufactured using Agilent's SureDesign web based tool and ordered from Agilent. Other probe design software and probe manufactures may also be used.

In certain aspects, large fragment capture comprises the following steps in the order shown:

1. DNA Isolation

Genomic DNA is obtained by standard isolation methods. These standard methods generally result in DNA of ˜10 kb to ˜100 kb in length. In some embodiments, the DNA may be sheared to produce somewhat smaller fragments.

2. DNA Capture

DNA is mixed with the custom probes described herein along with buffers commercially available for DNA capture. Fragments are incubated with standard temperature gradient protocols for hybridization of probes to DNA. Capture DNA is then mixed with capture beads commercially available and washed with buffers commercially available. Captured fragments are eluted and DNA size determined by standard bioanalyzer trace methods.

3. Shearing

Captured DNA fragments will then be sheared using mechanical or acoustic shearing instruments to sizes appropriate for the sequencing technology used for next generation sequencing (NGS). One method of sequencing is with an Illumina HiSeq instrument that requires 100-1000 bp fragments for sequencing. Newer sequencing technologies may require large fragments.

4. DNA Sequencing and Analysis

After shearing, DNA must be end-repaired and sequencing adapters ligated for currently available sequencing technologies. Standard reagents and protocols are available for this step. DNA is then sequenced. Sequencing results are aligned and genome coverage determined. Comparison between coverage of the targeted genes will be compared to coverage across the whole genome is done to demonstrate that the method with fewer probes and large fragments captured results in complete coverage the genes targeted.

The spacing between probes may alter capture efficiency due to the flexibility or rigidity of DNA and the secondary structure of the DNA that forms between probes. Probe spacing is required for multi intramolecular capture interactions to occur as probes are 80-120 bp in length in certain embodiments. In certain aspects, spacing between probes varies between 200 and 10,000 bp. In a preferred embodiment, spacing between probes is about 2000 bp.

When DNA is prepared or sheared, the ends of the DNA do not break evenly leaving overhangs of single stranded DNA. These overhangs may interfere with efficient capture as they may hybridize differently with probes. In some embodiments, the method further comprises performing end-repair after DNA isolation and before capture.

In some embodiments, DNA fragments of different size are generated for comparison of capture efficiency. Un-sheared DNA (˜10 Kb to 100 kb fragments), and DNA that has been sheared to various sizes such as ˜25 kb, ˜10 kb, ˜5 kb, ˜2 Kb, and the standard ˜200 bp fragment size may be used. The size of genomic DNA fragments may be determined by electrophoresis in an agarose gel according to standard methods and protocols.

In certain aspects, the present invention involves using large fragment capture to remove pseudogene sequences. The human genome contains about 22,000 actively transcribed and functional genes. In addition to these active genes, the human genome also contains thousands of pseudogenes. These are DNA sequences that resemble functional genes and have high homology to functional genes, but lack sequences to make them active genes. The similarity between functional genes and pseudogenes creates difficulty in capture efficiency and sequence alignment. Large fragment capture can be used to alleviate this challenge. In order for good discrimination between pseudogenes and active genes, probes must target regions with ˜30 bp different. Since homology is high between pseudogenes and their active counterparts, current small fragment capture methods requiring many overlapping probes cannot be designed to discriminate between pseudogenes and active genes at those homologous regions. With large fragment capture, probes can be designed to only unique pseudogene regions in comparison to their active gene counterparts. These probes can then be used to capture and remove large regions that extend into the homologous areas where distinguishing probes cannot be designed. Using such a design, entire pseudogenes can be selectively removed, leaving only active genes for sequencing. Alignment is improved as only sequences to active genes remain.

In another aspect, the present invention relates to using large fragment capture for genes of interest with high homology. Just as pseudogenes have high homology to functionally active genes, some active gene families have high homology between them. The same large fragment capture technique can be used to specifically capture genes that have large regions with high homology to other DNA sequences since probes can be designed to only regions with sufficient sequence differences yet capture the entire gene.

In some embodiments, the step of isolating genomic DNA from a biological sample results in the breaking of the genomic DNA into fragments of about 10 kb to about 100 kb, e.g., any range within about 10 kb to about 100 kb, such as about 10 kb to about 20 kb, about 10 kb to about 50 kb, about 10 kb to about 80 kb, about 20 kb to about 80 kb, about 40 kb to about 60 kb, etc. In certain aspects, the step of isolating genomic DNA from a biological sample comprises shearing the genomic DNA into fragments of about 2,000 bp to about 10,000 bp, e.g., any range within about 2,000 bp to about 10,000 bp such as about 2,000 bp to about 4,000 bp, about 2,000 bp to about 6,000 bp, about 2,000 to about 8,000 bp, about 4,000 bp to about 10,000 bp, etc.

In other aspects, the set of probes targets sequences across the large fragment of DNA at intervals of about 500 bp to about 10,000 bp, e.g. any range within about 500 bp to about 10,000 bp such as about 500 bp to about 5,000 bp, about 1,000 bp to about 4,000 bp, about 1,000 bp to about 2,000 bp, about 1,000 bp to about 8,000 bp, about 2,000 bp to about 6,000 bp, etc. In some embodiments the probes target sequences across the large fragment of DNA at intervals of about 500 bp, about 600 bp, about 700 bp, about 800 bp, about 900 bp, about 1,000 bp, about 1,100 bp, about 1,200 bp, about 1,300 bp, about 1,400 bp, about 1,500 bp, about 1,600 bp, about 1,700 bp, about 1,800 bp, about 1,900 bp, about 2,000 bp, about 2,100 bp, about 2,200 bp, about 2,300 bp, about 2,400 bp, about 2,500 bp, about 2,600 bp, about 2,700 bp, about 2,800 bp, about 2,900 bp, about 3,000 bp, about 3,500 bp, about 4,000 bp, about 4,500 bp, about 5,000 bp, about 5,500 bp, about 6,000 bp, about 6,500 bp, about 7,000 bp, about 7,500 bp, about 8,000 bp, about 8,500 bp, about 9,000 bp, about 9,500 bp, or about 10,000 bp. In certain embodiments the intervals are substantially regular intervals, the difference in the sizes of intervals being about 250 bp, about 200 bp, about 150 bp, about 100 bp, or about 50 bp of each other interval.

In certain aspects of the present invention, the first set of probes or second set of probes comprises probes of about 50 bp to about 500 bp in length, e.g., any range within about 50 bp to about 500 bp such as about 80 bp to about 120 bp, about 50 bp to about 250 bp, about 100 to about 500 bp, about 150 bp to about 300 bp, etc. In certain aspects, the probes comprise a first segment that hybridizes with the large fragment of DNA and a second segment that does not hybridize with the large fragment of DNA. The second segment may be about 20 bp, about 30 bp, about 40 bp, about 50 bp, about 60 bp, about 70 bp, about 80 bp, about 90 bp, or about 100 bp in length.

In some embodiments, the large fragment of DNA is about 2,000 bp to about 50,000 bp in length, e.g., any range within about 2,000 bp to about 50,000 bp such as about 2,000 bp to about 20,000 bp, about 2,000 bp to about 10,000 bp, about 2,000 bp to about 5,000 bp, about 5,000 bp to about 50,000 bp, about 10,000 bp to about 40,000 bp, etc.

The step of shearing the DNA may be accomplished by a number of methods. Such methods include sonication, needle shearing, nebulization, point-sink shearing and passage through a pressure cell. Restriction digest is the intentional laboratory breaking of DNA strands. It is an enzyme-based treatment used in biotechnology to cut DNA into smaller strands in order to study fragment length differences among individuals or for gene cloning. This method fragments DNA either by the simultaneous cleavage of both strands, or by generation of nicks on each strand of dsDNA to produce dsDNA breaks.

Acoustic shearing involves the transmission of high-frequency acoustic energy waves delivered to a DNA library. The transducer is bowl shaped so that the waves converge at the target of interest. Nebulization forces DNA through a small hole in a nebulizer unit, which results in the formation of a fine mist that is collected. Fragment size is determined by the pressure of the gas used to push the DNA through the nebulizer, the speed at which the DNA solution passes through the hole, the viscosity of the solution, and the temperature.

Sonication, a type of hydrodynamic shearing, subjects DNA to hydrodynamic shearing by exposure to brief periods of sonication. Point-sink shearing, a type of hydrodynamic shearing, uses a syringe pump to create hydrodynamic shear forces by pushing a DNA library through a small abrupt contraction. In some embodiments, about 90% of fragment lengths fall within a two-fold range.

Needle shearing creates shearing forces by passing DNA libraries through small gauge needle. The DNA passes through a gauge needle several times to physically tear the DNA into fine pieces. French pressure cells pass DNA through a narrow valve under high pressure to create high shearing forces. With a French press, the shear force can be carefully modulated by adjusting the piston pressure. The press provides a single pass through the point of maximum shear force, limiting damage to delicate biological structures due to repeated shear, as may occur in other disruption methods.

As used herein, the terms “nucleic acid” and “polynucleotide” are used interchangeably, and include polymeric forms of nucleotides of any length, either deoxyribonucleotides or ribonucleotides, or analogs thereof. The following are non-limiting examples of polynucleotides: a gene or gene fragment, exons, introns, messenger RNA (mRNA), microRNA transfer RNA (tRNA), ribosomal RNA (rRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The term also includes both double- and single-stranded molecules.

In some embodiments, the purified small fragments of DNA from the biological sample are analyzed by Sequencing by Synthesis (SBS) techniques. SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in some of the methods described herein, more than one type of nucleotide monomer can be provided to a target nucleic acid in the presence of a polymerase in a delivery.

SBS can utilize nucleotide monomers that have a terminator moiety or those that lack any terminator moieties. Methods utilizing nucleotide monomers lacking terminators include, for example, pyrosequencing and sequencing using γ-phosphate-labeled nucleotides. In methods using nucleotide monomers lacking terminators, the number of different nucleotides added in each cycle can be dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.). In preferred methods a terminator moiety can be reversibly terminating.

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.). It is also possible, however, to use the same label for the two or more different nucleotides present in a sequencing reagent or to use detection optics that do not necessarily distinguish the different labels. Thus, in a doublet sequencing reagent having a mixture of A/C both the A and C can be labeled with the same fluorophore. Furthermore, when doublet delivery methods are used all of the different nucleotide monomers can have the same label or different labels can be used, for example, to distinguish one mixture of different nucleotide monomers from a second mixture of nucleotide monomers. For example, using the [First delivery nucleotide monomers]+[Second delivery nucleotide monomers] nomenclature set forth above and taking an example of A/C+(1/T), the A and C monomers can have the same first label and the G and T monomers can have the same second label, wherein the first label is different from the second label. Alternatively, the first label can be the same as the second label and incorporation events of the first delivery can be distinguished from incorporation events of the second delivery based on the temporal separation of cycles in an SBS protocol. Accordingly, a low resolution sequence representation obtained from such mixtures will be degenerate for two pairs of nucleotides (T/G, which is complementary to A and C, respectively; and C/A which is complementary to G/T, respectively).

Some embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. No. 6,210,891; U.S. Pat. No. 6,258,568 and U.S. Pat. No. 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons.

In another example type of SBS, cycle sequencing is accomplished by stepwise addition of reversible terminator nucleotides containing, for example, a cleavable or photobleachable dye label as described, for example, in U.S. Pat. No. 7,427,67, U.S. Pat. No. 7,414,1163 and U.S. Pat. No. 7,057,026, the disclosures of which are incorporated herein by reference. This approach is being commercialized by Solexa (now Illumina Inc.), and is also described in WO 91/06678 and WO 07/123,744 (filed in the United States Patent and Trademark Office as U.S. Ser. No. 12/295,337), each of which is incorporated herein by reference in their entireties. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

In other embodiments, Ion Semiconductor Sequencing is utilized to analyze the purified small fragments of DNA from the sample. Ion Semiconductor Sequencing is a method of DNA sequencing based on the detection of hydrogen ions that are released during DNA amplification. This is a method of “sequencing by synthesis,” during which a complementary strand is built based on the sequence of a template strand.

For example, a microwell containing a template DNA strand to be sequenced can be flooded with a single species of deoxyribonucleotide (dNTP). If the introduced dNTP is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple dNTP molecules will be incorporated in a single cycle. This leads to a corresponding number of released hydrogens and a proportionally higher electronic signal.

This technology differs from other sequencing technologies in that no modified nucleotides or optics are used. Ion semiconductor sequencing may also be referred to as ion torrent sequencing, pH-mediated sequencing, silicon sequencing, or semiconductor sequencing. Ion semiconductor sequencing was developed by Ion Torrent Systems Inc. and may be performed using a bench top machine. Rusk, N. (2011). “Torrents of Sequence,” Nat Meth 8(1): 44-44. Although it is not necessary to understand the mechanism of an invention, it is believed that hydrogen ion release occurs during nucleic acid amplification because of the formation of a covalent bond and the release of pyrophosphate and a charged hydrogen ion. Ion semiconductor sequencing exploits these facts by determining if a hydrogen ion is released upon providing a single species of dNTP to the reaction.

For example, microwells on a semiconductor chip that each contain one single-stranded template DNA molecule to be sequenced and one DNA polymerase can be sequentially flooded with unmodified A, C, G or T dNTP. Pennisi, E. (2010). “Semiconductors inspire new sequencing technologies” Science 327(5970): 1190; and Perkel, J., “Making contact with sequencing's fourth generation” Biotechniques (2011). The hydrogen ion that is released in the reaction changes the pH of the solution, which is detected by a hypersensitive ion sensor. The unattached dNTP molecules are washed out before the next cycle when a different dNTP species is introduced.

Beneath the layer of microwells is an ion sensitive layer, below which is a hypersensitive ISFET ion sensor. All layers are contained within a CMOS semiconductor chip, similar to that used in the electronics industry. Each released hydrogen ion triggers the ISFET ion sensor. The series of electrical pulses transmitted from the chip to a computer is translated into a DNA sequence, with no intermediate signal conversion required. Each chip contains an array of microwells with corresponding ISFET detectors. Because nucleotide incorporation events are measured directly by electronics, the use of labeled nucleotides and optical measurements are avoided.

An example of a Ion Semiconductor Sequencing technique suitable for use in the methods of the provided disclosure is Ion Torrent sequencing (U.S. Patent Application Numbers 2009/0026082, 2009/0127589, 2010/0035252, 2010/0137143, 2010/0188073, 2010/0197507, 2010/0282617, 2010/0300559), 2010/0300895, 2010/0301398, and 2010/0304982), the content of each of which is incorporated by reference herein in its entirety. In Ion Torrent sequencing, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to a surface and are attached at a resolution such that the fragments are individually resolvable. Addition of one or more nucleotides releases a proton (H+), which signal detected and recorded in a sequencing instrument. The signal strength is proportional to the number of nucleotides incorporated. User guides describe in detail the Ion Torrent protocol(s) that are suitable for use in methods of the invention, such as Life Technologies' literature entitled “Ion Sequencing Kit for User Guide v. 2.0” for use with their sequencing platform the Personal Genome Machine™ (PCG).

In some embodiments, as a part of the sample preparation process, “barcodes” may be associated with each sample. In this process, short oligos are added to primers, where each different sample uses a different oligo in addition to a primer.

The term “library”, as used herein refers to a library of genome-derived sequences. The library may also have sequences allowing amplification of the “library” by the polymerase chain reaction or other in vitro amplification methods well known to those skilled in the art. The library may also have sequences that are compatible with next-generation high throughput sequencers such as an ion semiconductor sequencing platform.

In certain embodiments, the primers and barcodes are ligated to each sample as part of the library generation process. Thus during the amplification process associated with generating the ion amplicon library, the primer and the short oligo are also amplified. As the association of the barcode is done as part of the library preparation process, it is possible to use more than one library, and thus more than one sample. Synthetic DNA barcodes may be included as part of the primer, where a different synthetic DNA barcode may be used for each library. In some embodiments, different libraries may be mixed as they are introduced to a flow cell, and the identity of each sample may be determined as part of the sequencing process. Sample separation methods can be used in conjunction with sample identifiers. For example a chip could have 4 separate channels and use 4 different barcodes to allow the simultaneous running of 16 different samples.

By whole exome sequencing of individuals with a neuromuscular disease phenotype component and their family members, the inventors have made several discoveries of genetic mutations that result in a variety of neuromuscular diseases. These discoveries include identification of novel mutations in genes that have previously been associated with NMD as well as novel mutations in genes that have not previously been associated with NMD. These mutations are described in FIGS. 2 and 3.

In some embodiments, the present invention relates to diagnosing a subject with NMD by detecting a mutation in a gene with a variant of high confidence. High confidence variants (FIG. 2) are those variants that have high quality sequencing data and the variants identified are most likely causal. In these cases, additional evidence supports the conclusions such as additional families that the inventors have sequenced with mutations in the same gene and/or the phenotype is consistent with literature evidence for loss of function of the gene in which the variant is identified.

In some embodiments, the present invention relates to diagnosing a subject with NMD by detecting a mutation in a gene with a variant of low confidence. Variants of low confidence (FIG. 3) are from high quality sequencing results, but there is less supporting evidence for that variant being involved in disease. For instance, the variant may be in a gene that may be involved in NMD, but never before reported as disease causing. The variant might not be assigned a highly damaging value by prediction algorithms and no evidence is available to confirm damage to the gene product. It may be a variant that is likely the cause of disease but there is no clear biological tie to the disease as yet.

One type of NMD is spinal muscular atrophy (SMA). SMA is a currently untreatable, autosomal recessive genetic disease caused by a deficiency of full-length survival motor neuron (SMN) protein. The symptoms are the result of progressive degeneration of motor neurons in the anterior horn of the spinal cord resulting in weakness and wasting of the voluntary muscles.

Type I (Acute) SMA is also called Werdnig-Hoffmann Disease. SMA type I is evident before birth or within the first few months of life. There may be a reduction in fetal movement in the final months of pregnancy. There is a general weakness in the intercostals and accessory respiratory muscles. The chest may appear concave. Symptoms include floppiness of the limbs and trunk, feeble movements of the arms and legs, swallowing and feeding difficulties, and impaired breathing. Affected children never sit or stand and usually die before the age of 2.

Type II (Chronic) SMA is usually diagnosed by 15 months. Children may have respiratory problems, floppy limbs, decreased or absent deep tendon reflexes, and twitching of arm, leg, or tongue muscles. These children may learn to sit but cannot stand or walk. Life expectancy varies. Feeding and swallowing problems are not usually characteristic of Type II, although in some patients a feeding tube may become necessary. Tongue fasciculations are less often found in children with Type II but a fine tremor in the outstretched fingers is common.

Type III (Mild) SMA, often referred to as Kugelberg-Welander or Juvenile Spinal Muscular Atrophy, is usually diagnosed between 2 and 17 years of age. Symptoms include abnormal manner of walking; difficulty running, climbing steps, or rising from a chair; and slight tremor of the fingers. The patient with Type DI can stand alone and walk; tongue fasciculations are seldom seen. Types I, II and III progress over time, accompanied by deterioration of the patient's condition.

Type IV (Adult Onset) typically begins after age 35. Adult SMA is characterized by insidious onset and very slow progression. The bulbar muscles are rarely affected in Type IV. It is not clear that Type IV SMA is etiologically related to the Type I-III forms. There is a second type of Adult Onset X-Linked SMA, known as Kennedy's Syndrome or Bulbo-Spinal Muscular Atrophy. It occurs only in males, and, unlike the other forms of SMA, it is associated with a mutation in the gene that codes for part of the androgen receptor. The facial and tongue muscles are noticeably affected. The course of the Adult Onset disease is variable, but in general it tends to be slowly progressive or nonprogressive.

Type I, II and III SMA are caused by a mutation in a part of the DNA called the survival motor neuron (SMN1) gene, which normally produces a protein called SMN. Because of their gene mutation, people with SMA make less SMN protein, which results in the loss of motor neurons. SMA symptoms may be improved by increasing the levels of SMN protein. Normally the SMN1 gene provides instructions for making a protein called Survival of Motor Neuron 1. The SMN1 protein helps to assemble the cellular machinery needed to process pre-mRNA. More than 90 percent of individuals with spinal muscular atrophy lack part or all of both copies of the SMN1 gene. A small percentage of people with this condition lack one copy of the SMN1 gene and have a small type of mutation in the remaining copy. About 30 different mutations have been identified. The most frequent of these mutations replaces the amino acid tyrosine with cysteine at position 272 in the SMN1 protein. Other mutations replace amino acids at different positions or produce an abnormally short protein. As a result of these missing or altered genes, cells have a shortage of functional SMN1 protein. It remains unclear why motor neurons are particularly vulnerable to a shortage of this protein. Loss of the SMN1 protein from motor neurons results in the degeneration of these nerve cells, leading to the signs and symptoms of spinal muscular atrophy.

In some cases of spinal muscular atrophy, particularly the milder cases, the SMN1 gene is replaced by an almost identical gene called SMN2. Typically, people who do not have spinal muscular atrophy have two copies of the SMN2 gene. In some affected individuals, however, the SMN2 gene replaces the SMN1 gene, and as a result, the number of SMN2 genes increases from two to three or more (and the number of SMN1 genes decreases). On a limited basis, extra SMN2 genes can help replace the protein needed for the survival of motor neurons. In general, symptoms are less severe and begin later in life in affected individuals with three or more copies of the SMN2 gene. The SMN2 gene provides instructions for making a protein called survival of motor neuron 2. This protein is made in four different versions, but only isoform d is full size and functional and appears to be identical to the SMN1 protein. The other isoforms (a, b, and c) are smaller and may not be fully functional. It appears that only a small amount of the protein made by the SMN2 gene is isoform d. Among individuals with spinal muscular atrophy (who lack functional SMN1 genes), additional copies of the SMN2 gene can modify the course of the disorder. On a limited basis, the extra SMN2 genes can help replace the protein needed for the survival of motor neurons. Spinal muscular atrophy still occurs, however, because most of the proteins produced by SMN2 genes are isoforms a, b, and c, which are smaller than the SMN1 protein and cannot fully compensate for the loss of SMN1 genes. A recent article by Cartegni and Krainer [Nature Genetics 30, 377-384 (2002)] suggests that the molecular basis for the failure of the nearly identical gene SMN2 to provide full protection against SMA stems from inefficient recognition of an exonic splicing enhancer by the splicing factor SF2/ASF. Even so, the small amount of full-sized protein produced from three or more copies of the SMN2 gene can delay onset and produce less severe symptoms, as seen in spinal muscular atrophy, types II and III.

Another form of NMD is myotonic dystrophy. Myotonic dystrophy (DM) is an autosomal dominant neuromuscular disease which is the most common form of muscular dystrophy affecting adults. The clinical picture in DM is well established but exceptionally variable. Although generally considered a disease of muscle, with myotonia, progressive weakness and wasting, DM is characterized by abnormalities in a variety of other systems. DM patients often suffer from cardiac conduction defects, smooth muscle involvement, hypersomnia, cataracts, abnormal glucose response, and, in males, premature balding and testicular atrophy. The mildest form, which is occasionally difficult to diagnose, is seen in middle or old age and is characterized by cataracts with little or no muscle involvement. The classical form, showing myotonia and muscle weakness, most frequently has onset in early adult life and in adolescence. The most severe form, which occurs congenitally, is associated with generalized muscular hypoplasia, mental retardation, and high neonatal mortality.

Myotonic dystrophy type 1 (DM1) is caused by a trinucleotide (CTG) expansion (n=50 to >3000) in the 3′-untranslated region (3′UTR) of the Dystrophia myotonica-protein kinase (DMPK) gene. Myotonic dystrophy type 2 (DM2) is caused by a tetranucleotide (CCTG)ₙ expansion (n=75 to about 11,000) in the first intron of zinc finger protein 9 (ZNF9) gene (Ranum, et al., 2002, Curr. Opin. in Genet. and Dev. 12:266-271). There appears to be a common pathogenic mechanism involving the accumulation of transcripts into discrete nuclear RNA foci containing long tracts of CUG or CCUG repeats expressed from the expanded allele, and both DM1 and DM2 mutant transcripts accumulate as foci within muscle nuclei (Liguori, et al., 2001, Science 293: 864-867). Transgenic mice which express a large CTG repeat in the 3′-UTR of a human skeletal actin transgene develop myonuclear RNA foci, myotonia, and degenerative muscle changes similar to those seen in human DM (Mankodi, et al., 2000, Science 289: 1769-1773). The myotonia in such transgenic mice is caused by loss of skeletal muscle chloride (ClC-1) channels due to aberrant pre-mRNA splicing (Mankodi, et al., 2002, Mol. Cell. 10: 35-44). Similar ClC-1 splicing defects exist in DM1 and DM2.

The terms “treatment”, “treating”, and the like are used herein to generally mean obtaining a desired pharmacologic and/or physiologic effect. The effect may be prophylactic in terms of completely or partially preventing a disease, condition, or symptoms thereof, and/or may be therapeutic in terms of a partial or complete cure for a disease or condition and/or adverse effect attributable to the disease or condition. “Treatment” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition (e.g., arresting its development); or (c) relieving the disease or condition (e.g., causing regression of the disease or condition, providing improvement in one or more symptoms). For example, “treatment” of DM1 and DM2 encompasses a complete reversal or cure of the disease, or any range of improvement in conditions and/or adverse effects attributable to DM1 and DM2. Merely to illustrate, “treatment” of DM1 and DM2 includes an improvement in any of the following effects associated with DM1, DM2 or combination thereof: muscle weakness, muscle wasting, grip strength, cataracts, difficulty relaxing grasp, irregularities in heartbeat, constipation and other digestive problems, retinal degeneration, low IQ, cognitive defects, frontal balding, skin disorders, atrophy of the testicles, insulin resistance and sleep apnea. Improvements in any of these conditions can be readily assessed according to standard methods and techniques known in the art. Other symptoms not listed above may also be monitored in order to determine the effectiveness of treating DM1 or DM2. The population of subjects treated by the method of the disease includes subjects suffering from the undesirable condition or disease, as well as subjects at risk for development of the condition or disease.

By the term “therapeutically effective dose” is meant a dose that produces the desired effect for which it is administered. The exact dose will depend on the purpose of the treatment, and will be ascertainable by one skilled in the art using known techniques (see, e.g., Lloyd (1999) The Art, Science and Technology of Pharmaceutical Compounding).

In some embodiments of the present invention, the methods further comprise treating NMD in a subject. Treating NMD may be accomplished by appropriate therapies targeted to the particular form of NMD and the accompanying symptoms. Examples of such therapies include, but are not limited to, laminin-111 protein therapy, which works to stabilize the sarcolemma and reduce muscle degeneration. In some examples, a source of muscle cells can be added to aid in muscle regeneration and repair. In some aspects of the present disclosure, satellite cells are administered to a subject in combination with laminin therapy. U.S. Patent Publication 2006/0014287, incorporated by reference herein to the extent not inconsistent with the present disclosure, provides methods of enriching a collection of cells in myogenic cells and administering those cells to a subject. In further aspects, stem cells, such as adipose-derived stem cells, are administered to the subject. Suitable methods of preparing and administering adipose-derived stem cells are disclosed in U.S. Patent Publication 2007/0025972, incorporated by reference herein to the extent not inconsistent with the present disclosure. Additional cellular materials, such as fibroblasts, can also be administered, in some examples.

Additional therapeutic agents include α7β1 modulatory agents and agents which enhance α7β1 modulatory agents, such as a component of the extracellular matrix, such as an integrin, dystrophin, dystroglycan, utrophin, or a growth factor. In some examples, the additional therapeutic agent reduces or enhances expression of a substance that enhances the formation or maintenance of the extracellular matrix. In some examples, the additional substance can include aggrecan, angiostatin, cadherins, collagens (including collagen I, collagen III, or collagen IV), decorin, elastin, enactin, endostatin, fibrin, fibronectin, osteopontin, tenascin, thrombospondin, vitronectin, and combinations thereof. Biglycans, glycosaminoglycans (such as heparin), glycoproteins (such as dystroglycan), proteoglycans (such as heparan sulfate), and combinations thereof can also be administered.

In some embodiments, growth stimulants such as cytokines, polypeptides, and growth factors such as brain-derived neurotrophic factor (BDNF), CNF (ciliary neurotrophic factor), EGF (epidermal growth factor), FGF (fibroblast growth factor), glial growth factor (GGF), glial maturation factor (GMF) glial-derived neurotrophic factor (GDNF), hepatocyte growth factor (HGF), insulin, insulin-like growth factors, kerotinocyte growth factor (KGF), nerve growth factor (NGF), neurotropin-3 and -4, PDGF (platelet-derived growth factor), vascular endothelial growth factor (VEGF), and combinations thereof may be administered with one of the disclosed methods.

Other therapeutic interventions can include, but are not limited to, proteins, peptides, polypeptides, antibodies, stem cells, nucleic acids, polynucleotides, oligonucleotides, exercise regimens, nutritional supplements, or small molecules. The therapeutic intervention can be pharmaceutical compositions currently approved by the FDA for other indications or compositions comprising a new chemical entity. In one embodiment, the therapeutic intervention is a compound or biologic selected from a compound or biologic library, such as a combinatorially-generated library. Combinatorial approaches are amenable to the development of a large number of potential therapeutics that are created by second, third, and fourth generation compounds modeled on active, but otherwise undesirable compounds. In one embodiment, the therapeutic intervention increases expression of SMN protein. Such therapeutic interventions can include, but are not limited to, compounds such as valproic acid, phenylbutyrate, sodium butyrate, hydroxyurea, trapoxin, and trichostatin A as well as other types of therapeutic interventions. In another embodiment, the therapeutic intervention has no effect on expression of SMN protein.

The present invention is further illustrated by the following examples that should not be construed as limiting. The contents of all references, patents, and published patent applications cited throughout this application, as well as the Figures, are incorporated herein by reference in their entirety for all purposes.

### EXAMPLES

### Example 1

### Advancing Genetic Diagnosis of Infantile Forms of Spinal Muscular Atrophy

### Background and Objectives

Neuromuscular disorders are among the most common form of inherited childhood disorders with prevalence as high as 1 in 1700. The inventors have focused on rare lethal infantile neuromuscular disorders similar to Type I SMA, but negative for SMN1 mutations. Their long-term efforts identified the first disease-associated mutations in UBA1 [X-linked lethal infantile spinal muscular atrophy (XL-SMA); MIM 3018300] (Ramser et al, 2008). They have identified and collected samples from numerous families and isolated male cases suspected of having XL-SMA and screened them for mutations in the UBA1 gene by sequencing. SMN1 and UBA1 mutation negative cases are being further evaluated by exome sequencing to identify novel disease causing mutations.

### Results

The inventors developed a custom Ion Torrent AmpliSeq panel to sequence all 26 exons of UBA1. To date, the UBA1 locus has been evaluated in 24 suspected X-linked probands and family members. All of these cases are UBA1 mutation negative. All variants identified in the coding regions of UBA1 in these cases were previously identified in variant databases, are present in the general population at relatively abundant frequencies, and are not associated with disease. For further evaluation of these cases, the inventors have sequenced exomes of 5 affected individuals and their appropriate relatives from 3 separate pedigrees as well as 6 affected singleton cases and identified potential disease causing mutations. Of particular interest, they identified novel compound heterozygous mutations in two affected siblings in CHRND (acetylcholine receptor, muscle, delta subunit; OMIM 100720). Both affected boys inherited a frameshift mutation from one parent and a missense mutation from the other parent. The missense mutation is in the highly conserved cys-loop domain of CHRND that regulates gating speed. CHRND mutations have been previously associated with Multiple Pterygium Syndrome (Lethal Type) and congenital forms of myasthenic syndrome. In a family with a strong pattern of X-linked inheritance, they identified a novel start loss M1V mutation in SCML2 (sex comb on midleg, drosophila, homolog-like 2; OMIM 300208). SCML2 binds histone peptides that are monomethylated at lysine residues and is part of the polycomb repressive complex 1 that regulates developmental genes. Human mutations have not been previously reported in SCML2. Other potential disease causing mutations are also presented (See FIGS. 2 and 3).

### CONCLUSIONS

The findings demonstrate that infantile forms of SMA can be caused by divers mutations yet result in similar phenotypes. The inventors describe here novel mutations in a known disease causing gene, CHRND, as well as mutations in novel genes that have not been described previously. The results demonstrate the utility of exome sequencing to identify causes of rare and severe childhood disorders. With current sequencing technologies, molecular diagnoses can be acquired in a timely manner and provides patients, families and physicians with tools to find ways to understand and possibly treat devastating childhood diseases.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

The list of sequences are assigned the following SEQ ID NOS for convenience and for reference to the Sequence Listing:

SCML2 (SEQ ID NO:1) is Genomic DNA sequence; X DNA:chromosome chromosome:GRCh37:X: 18257434: 18372847:1;

SCML2 (SEQ ID NO:2) is a Protein Amino Acid Sequence (700aa).

CHRND (SEQ ID NO:3) is a Genomic DNA Sequence; 2 DNA:chromosome chromosome:GRCh37:2:233390703:233401377:1.

CHRND (SEQ ID NO:4) is Protein Amino Acid Sequence (517aa).

OFD1 (SEQ ID NO:5) is Genomic DNA Sequence; X DNA:chromosome chromosome:GRCh37:X: 13752832: 13787480:1.

OFD1 (SEQ ID NO:6) is a Protein Amino Acid Sequence (1,012aa).

DYNC1H1 (SEQ ID NO:7) is a Genomic DNA Sequence; 14 DNA:chromosome chromosome:GRCh37: 14:102430865:102517129:1.

DYNC1H1 (SEQ ID NO:8) is a Protein Amino Acid Sequence (4,646aa).

COL6A3 (SEQ ID NO:9 is a Genomic DNA Sequence; 2 DNA:chromosome chromosome:GRCh37:2:238232646:238323018:1.

COL6A3 (SEQ ID NO: 10) is a Protein Amino Acid Sequence (3,177aa).

EMD (SEQ ID NO: 11) is a Genomic DNA Sequence; X DNA:chromosome chromosome:GRCh37:X: 153607557:153609883:1.

EMD (SEQ ID NO: 12) is a Protein Amino Acid Sequence (254aa).

ARHGAP4 (SEQ ID NO: 13) is a Genomic DNA Sequence; X DNA:chromosome chromosome:GRCh37:X:153172821:153200452:1.

ARHGAP4 (SEQ ID NO: 14) is a Protein Amino Acid Sequence (946aa).

FLNA (SEQ ID NO: 15) is a Genomic DNA Sequence; X DNA:chromosome chromosome:GRCh37:X:153576892:153603006:1.

FLNA (SEQ ID NO: 16) is a Protein Amino Acid Sequence (2,647aa).

MID1IP1 (SEQ ID NO: 17) is a Genomic DNA Sequence; X DNA:chromosome chromosome: GRCh37:X:38660685:38665790:1.

MID1IP1 (SEQ ID NO: 18) is a Protein Amino Acid Sequence (183aa).

MID1 (SEQ ID NO: 19) is a Genomic DNA Sequence; X DNA:chromosome chromosome:GRCh37:X:10413350:10851773:1.

MID1 (SEQ ID NO: 20) is a Protein Amino Acid Sequence (667aa).

CFP (SEQ ID NO: 21) is a Genomic DNA Sequence; X DNA:chromosome chromosome:GRCh37:X:47483612:47489704:1.

CFP (SEQ ID NO: 22) is a Protein Amino Acid Sequence (469aa).

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed is:

1. A method of sequencing a large fragment of DNA, the method comprising:
a) isolating genomic DNA from a biological sample;
b) hybridizing the genomic DNA with a set of probes to form genomic DNA-probe complexes, wherein the set of probes targets sequences across the large fragment of DNA at intervals;
c) purifying the genomic DNA from the complexes with affinity chromatography;
d) shearing the genomic DNA to produce small fragments of DNA, wherein the small fragments of DNA comprise coding and non-coding sequences from the large fragment of DNA; and
e) sequencing the small fragments of DNA with Next Generation Sequencing (NGS) to obtain the sequence of the large fragment of DNA.

2. The method of claim 1, wherein the intervals across the large fragment of DNA are about 500 bp to about 5,000 bp.

3. The method of claim 2, wherein the intervals are regular intervals, the regular intervals being about 1,000 bp to about 2,000 bp.

4. The method of claim 3, wherein the regular intervals across the large fragment of DNA are about 2,000 bp.

5. The method of claim 1, wherein the large fragment of DNA is about 2,000 bp to about 50,000 bp in length.

6. The method of claim 1, wherein the set of probes comprises probes of about 80 bp to about 120 bp in length.

7. The method of claim 1, wherein the large fragment of DNA includes at least one gene.

8. The method of claim 7, wherein the at least one gene is associated with a neuromuscular disease (NMD).

9. The method of claim 1, wherein the large fragment of DNA includes at least fifty genes.

10. The method of claim 1, wherein the set of probes comprise an affinity tag selected from the group consisting of biotin and streptavidin.

11. The method of claim 1, wherein shearing the genomic DNA comprises sonication, needle shearing, restriction digest, acoustic shearing, nebulization, point-sink shearing, or passage through a pressure cell of the genomic DNA.

12. The method of claim 11, wherein shearing the genomic DNA comprises sonication.

13. The method of claim 1, further comprising performing end-repair of the genomic DNA after isolating the genomic DNA, wherein the end-repair prevents overhanging, single-stranded DNA from interfering with the hybridizing of the genomic DNA with the set of probes.

14. The method of claim 1, wherein the NGS comprises ion semiconductor sequencing, cycle sequencing, pyrosequencing, or sequencing using γ-phosphate-labeled nucleotides.

15. A method of sequencing a large fragment of DNA, the method comprising:
a) isolating genomic DNA from a biological sample;
b) hybridizing the genomic DNA with a first set of probes to form genomic DNA-probe complexes with a portion of the genomic DNA encoding a pseudogene, wherein the set of probes targets sequences across the pseudogene at intervals;
c) removing the portion of the genomic DNA encoding the pseudogene with affinity chromatography;
d) hybridizing the genomic DNA with a second set of probes to form genomic DNA-probe complexes, wherein the second set of probes targets sequences across the large fragment of DNA at intervals;
e) purifying the genomic DNA from the complexes with affinity chromatography;
f) shearing the genomic DNA to produce small fragments of DNA, wherein the small fragments of DNA comprise coding and non-coding sequences from the large fragment of DNA; and
g) sequencing the small fragments of DNA with Next Generation Sequencing (NGS) to obtain the sequence of the large fragment of DNA.

16. The method of claim 15, wherein the intervals across the pseudogene and/or the large fragment of DNA are about 500 bp to about 5,000 bp.

17. The method of claim 15, wherein the intervals across the pseudogene and/or the large fragment of DNA are regular intervals of about 2,000 bp.

18. The method of claim 15, wherein the large fragment of DNA is about 2,000 bp to about 50,000 bp in length.

19. The method of claim 15, wherein the large fragment of DNA includes at least one gene.

20. The method of claim 19, wherein the pseudogene and the at least one gene share at least 80% sequence homology.

21. A method of diagnosing a neuromuscular disease (NMD) in a subject, the method comprising:
a) obtaining a biological sample from the subject;
b) isolating genomic DNA from the biological sample;
c) sequencing in the genomic DNA at least one gene selected from the group consisting of SCML2, CHRND, OFD1, DYNC1H1, COL6A3, EMD, ARHGAP4, FLNA, MID1IP1, MID1, and CFP; and
d) diagnosing NMD in the subject if there is a mutation in the at least one gene.

22. The method of claim 21, wherein the mutation is selected from the group consisting of ASN76SER in SCML2, MET1VAL start loss in SCML2, ASP161ASN in CHRND, a single DNA base pair frameshift deletion at genomic position chromosome 2 position 233398958 in CHRND, GLU958LYS in OFD1, TRP1208LEU in DYNC1H1, LYS2483GLU in COL6A3, a DNA substitution of G to T at the splice junction of EMD at genomic position chromosome X position 153608155, PRO635LEU in ARHGAP4, VAL584LEU in FLNA, ARG655HIS in FLNA, ASP51ASN in MID1IP1, PRO667LEU in MID1, and CYS337TYR in CFP.
